(19) United States
(12) Patent Application Publication
(10) Pub. No.: US 2025/0072972 A1
(43) Pub. Date: Mar. 6, 2025

(54) METHOD FOR DETERMINING PLANE IN THREE-DIMENSIONAL SHAPE AND ELECTRONIC DEVICE PERFORMING THE SAME

(71) Applicant: Medipixel, Inc., Seoul (KR)

(72) Inventors: Donghyun WON, Seoul (KR); Eunjin JEONG, Seoul (KR); Donggyu KIM, Seoul (KR); Yeongjun KIM, Seoul (KR)

(73) Assignee: Medipixel, Inc., Seoul (KR)

(21) Appl. No.: 18/822,873

(22) Filed: Sep. 3, 2024

(30) Foreign Application Priority Data
Sep. 6, 2023 (KR) 10-2023-0118083
Apr. 19, 2024 (KR) 10-2024-0052755
Aug. 5, 2024 (KR) 10-2024-0104026

(51) Int. Cl. A61B 34/10; G16H 20/40; G16H 50/50

(52) U.S. Cl. CPC A61B 34/10; G16H 20/40; G16H 50/50; A61B 2034/105

(57) ABSTRACT

Provided is a method for determining a plane in three-dimensional shape to obtain precise size information of an aneurysm. The method includes generating an aneurysm region including: generating a vessel mesh; obtaining at least one input point; generating a separating plane based on the at least one input point; and generating the aneurysm region based on the separating plane.

### CROSS-REFERENCE TO RELATED APPLICATIONS

This application claims priority to and the benefit thereof under 35 U.S.C. § 119 to Korean Patent Application No. 10-2023-0118083 filed in the Korean Intellectual Property Office on Sep. 6, 2023, Korean Patent Application No. 10-2024-0052755 filed in the Korean Intellectual Property Office on Apr. 19, 2024, and Korean Patent Application No. 10-2024-0104026 filed in the Korean Intellectual Property Office on Aug. 5, 2024, the entire contents of which are incorporated herein by reference.

### BACKGROUND

### (a) Field

The disclosure relates to a method for determining a plane in three-dimensional shape and an electronic device performing the same.

### (b) Description of the Related Art

An aneurysm is a disease where part of an artery wall weakens and balloons out, which can occur in various forms such as cerebral aneurysm, aortic aneurysm, renal artery aneurysm, and splenic artery aneurysm. Among the treatment methods for aneurysms, coil embolization is a prominent technique where a thin coil is inserted into the aneurysm to block the flow of blood into the cerebral aneurysm.

To perform coil embolization effectively, it is crucial to accurately measure the size of the aneurysm, including volume and length, as these measurements are used to determine the appropriate amount of coil needed. Traditionally, medical professionals estimated the size of the aneurysm by analyzing images and using an ellipsoid etc., to approximate the size. However, there is an increasing demand for methods that can determine the size more precisely, ensuring it matches the actual shape of the aneurysm.

### SUMMARY

Some embodiments may provide a method for determining a plane in three-dimensional shape and an electronic device performing the same to obtain precise size information of an aneurysm.

According to an aspect of an embodiment, a method for generating an aneurysm region may include: generating a vessel mesh; obtaining at least one input point; generating a separating plane based on the at least one input point; and generating the aneurysm region based on the separating plane.

According to an aspect of an embodiment, an electronic device may include a processor; and a memory connected to the processor, wherein the memory is configured to store a program, the processor is configured to execute the program, and when the program is executed, the steps of a method for generating an aneurysm region are implemented.

Additional aspects may be set forth in part in the description which follows and, in part, may be apparent from the description, and/or may be learned by practice of the presented embodiments.

### BRIEF DESCRIPTION OF THE DRAWINGS

The above and other aspects, features, and advantages of certain embodiments of the present disclosure will become apparent from the following description taken in conjunction with the accompanying drawings, in which:

FIG. 1 is a schematic block diagram of the computing system according to an embodiment.

FIG. 2 is an example flowchart showing a method for generating aneurysm region by an electronic device according to an embodiment.

FIG. 3 illustrates a method of generating a separating plane on a vessel mesh by an electronic device according to an embodiment.

FIG. 4 illustrates a method of generating a plane by an electronic device according to an embodiment.

FIG. 5 illustrates a method of generating an aneurysm region on a vessel mesh by an electronic device according to an embodiment.

FIG. 6 is a flowchart of a method for generating an aneurysm region by an electronic device according to an embodiment.

FIGS. 7 and 8 illustrate a method for generating an aneurysm region on a vessel mesh by an electronic device according to an embodiment.

FIG. 9 is a flowchart illustrating a method for generating a separating plane on a vessel mesh by an electronic device according to an embodiment.

FIGS. 10 and 11 are illustrate a method for generating a separating plane on a vessel mesh by an electronic device according to an embodiment.

FIG. 12 is a flowchart illustrating a method for generating a separating plane by an electronic device according to an embodiment.

FIG. 13 is a flowchart illustrating a method for generating a separating plane by an electronic device according to an embodiment.

FIGS. 14 and 15 illustrate a method for generating a separating plane by an electronic device according to an embodiment.

FIG. 16 is a graph showing the length of the intersection line according to a rotation angle of a separating plane according to an embodiment.

FIG. 17 illustrate a method for generating a separating plane by an electronic device according to an embodiment.

### DETAILED DESCRIPTION OF THE INVENTION

In the following detailed description, only certain embodiments of the present invention have been shown and described, simply by way of illustration. As those skilled in the art would realize, the described embodiments may be modified in various different ways, all without departing from the spirit or scope of the present invention.

The drawings and description are to be regarded as illustrative in nature and not restrictive. Like reference numerals designate like elements throughout the specification. The sequence of operations or steps is not limited to the order presented in the claims or figures unless specifically indicated otherwise. The order of operations or steps may be changed, several operations or steps may be merged, a certain operation or step may be divided, and a specific operation or step may not be performed.

As used herein, the singular forms “a” and “an” are intended to include the plural forms as well, unless the context clearly indicates otherwise. Although the terms first, second, and the like may be used herein to describe various elements, components, steps and/or operations, these terms are only used to distinguish one element, component, step or operation from another element, component, step, or operation.

As used herein, each of such phrases as “A or B,” “at least one of A and B,” “at least one of A or B,” “A, B, or C,” “at least one of A, B, and C,” and “at least one of A, B, or C,” may include any possible combination of the items enumerated together in a corresponding one of the phrases.

Reference throughout the present disclosure to “one embodiment,” “an embodiment,” “an example embodiment,” or similar language may indicate that a particular feature, structure, or characteristic described in connection with the indicated embodiment is included in at least one embodiment of the present solution. Thus, the phrases “in one embodiment”, “in an embodiment,” “in an example embodiment,” and similar language throughout this disclosure may, but do not necessarily, all refer to the same embodiment.

Hereinafter, various embodiments of the present disclosure are described with reference to the accompanying drawings.

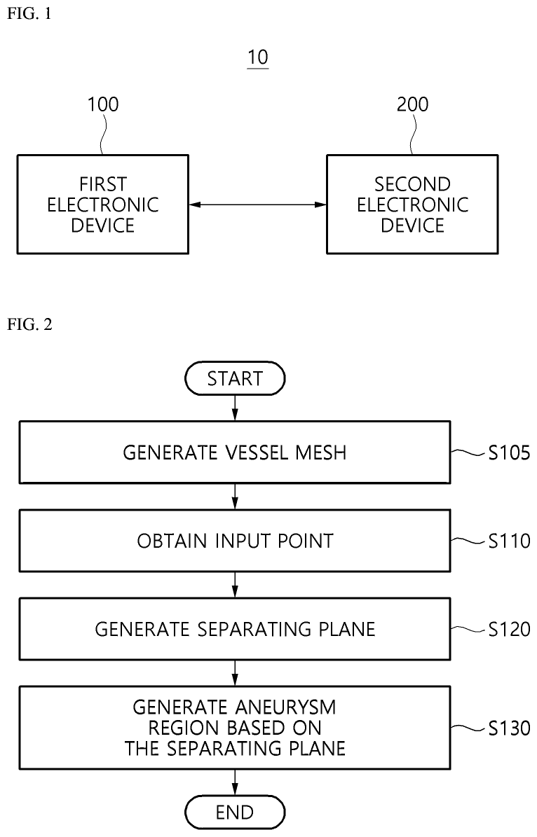

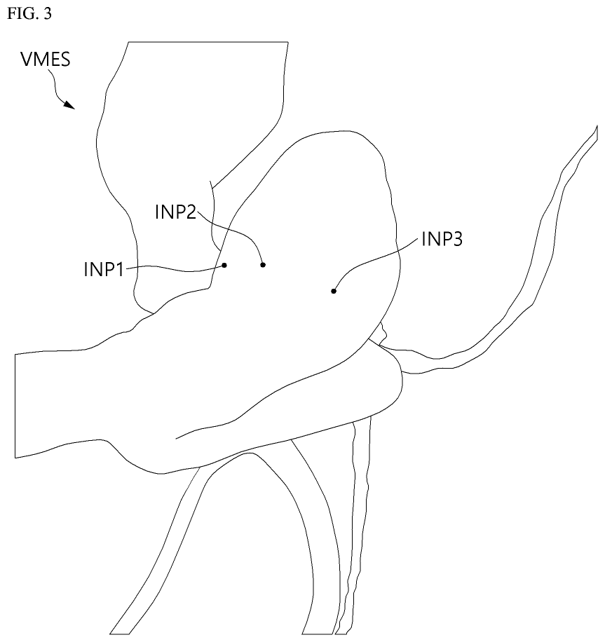

FIG. 1 is a schematic block diagram of the computing system according to an embodiment, and FIG. 2 is an example flowchart showing a method for generating aneurysm region by an electronic device according to an embodiment.

Referring to FIG. 1, a computing system 10 according to an embodiment may obtain a vessel image of a first user, perform image processing on the obtained vessel image, and display the processed image. For example, the computing system 10 may generate and display date in three-dimensional shape based on the obtained vessel image. The computing system 10 may provide the processed image to a second user. For example, the first user may be a patient, and the second user may be a medical professional.

In some embodiments, the computing system 10 may capture a brain vascular of the first user to obtain a brain vascular image and generate an aneurysm region by performing image processing on the brain vascular image. However, the embodiments are not limited thereto, and the computing system 10 may capture blood vessels that are the subject of angiography, such as the cardiovascular system or gastrointestinal vessels.

The computing system 10 according to an embodiment includes a first electronic device 100 and a second electronic device 200. The first electronic device 100 may be an imaging device that captures an image of the first user to obtain an image. For example, the first electronic device 100 may be an angiography equipment (for example, angio-device), Optical Coherence Tomography (OCT) equipment, Computed Tomography (CT) equipment, Magnetic Resonance Imaging (MRI) equipment, or Magnetic Resonance Angiography (MRA) equipment.

The first electronic device 100 may capture the first user from multiple imaging points to obtain the plurality of images. In some embodiments, the first electronic device 100 may capture the first user while rotating around the first user. In other embodiments, the first electronic device 100 may rotate the first user and capture the first user while rotating. The first electronic device 100 may transmit the obtained images to the second electronic device 200.

The second electronic device 200 may be a computing device performing image processing on a plurality of images received from the first electronic device 100. The second electronic device 200 may generate an aneurysm region based on the plurality of images. The second electronic device 200 may be a server, a data center, an Artificial Intelligence (AI) device, a Personal Computer (PC), a laptop computer, a mobile phone, a smart phone, a tablet PC, a wearable device, a healthcare device, etc.

In some embodiments, when the second electronic device 200 is implemented as a server, data center, or similar system, the computing system 10 may further include a third electronic device for interacting with the second user. For example, the third electronic device may interact with the second user by displaying images and receiving input from the second user. The third electronic device may communicate with the first electronic device and/or the second electronic device.

The second electronic device 200 may perform a method for determining a separating plane shown in FIG. 2. The determined separating plane is used to separate a parent artery and the aneurysm region in a vessel mesh, and the method for determining the separating plane of FIG. 2 can also be understood as a method for generating the aneurysm region. The second electronic device 200 may include a memory that stores a program for executing the method for determining the separating plane and a processor configured to execute the program to generate the aneurysm region. The second electronic device 200 may further include input/output devices (such as input devices like a mouse or keyboard, output devices like a display panel, and input/output devices like a touchscreen panel, etc.), communication devices, and others.

Referring to FIGS. 1 and 2, the second electronic device 200 may generate a vessel mesh (S105). The second electronic device 200 may receive a plurality of images captured by the first electronic device 100. The second electronic device 200 may generate the vessel mesh by applying marching cube algorithm to the plurality of images. The vessel mesh may be data representing blood vessels in three dimensions. The second electronic device 200 may display the vessel mesh through a display. The second user can observe the vessel mesh while changing viewpoint. For example, the second user can change the viewpoint by moving and/or rotating the vessel mesh while viewing the vessel mesh through the display.

The second electronic device 200 may obtain an input point (S110). The second user may input a point into the second electronic device 200 using an input/output device. The second electronic device 200 may generate a straight line toward the point from the viewpoint where the current vessel mesh is displayed, and determine the point where the straight line first contacts the vessel mesh as the input point.

When the second user inputs two or more points, the user may input the points while maintaining or changing the viewpoint. In an example, the second user may input a first point and a second point into the second electronic device 200 while viewing the vessel mesh from the first viewpoint. In another example, the second user may input the first point while viewing the vessel mesh from the first viewpoint, then change the viewpoint to the second viewpoint and input the second point. In this case, the second user may change the viewpoint by moving and/or rotating the vessel mesh. In the same manner as the aforementioned examples, the second user may also input three or more points.

The second electronic device 200 may generate a separating plane based on the input points (S120). In some embodiments, if the second user inputs three points into the second electronic device 200, the second electronic device 200 may generate the separating plane based on the three input points. In some embodiments, if the second user inputs two points into the second electronic device 200, the second electronic device 200 may generate the separating plane based on the two input points and a feature point. In some embodiments, the feature point may refer to a point related to the vessel mesh, such as an aneurysm entry point or a camera viewpoint. In some embodiments, if the second user inputs one point into the second electronic device 200, the second electronic device 200 may generate the separating plane based on the one input point and an aneurysm vector. The second electronic device 200 may generate a plane passing through the input point, with the aneurysm vector as a normal vector, as the separating plane. The aneurysm vector may be a vector directed from the aneurysm entry point to an aneurysm point. The aneurysm point may be a point located on an aneurysm region, determined based on user input or inference from an artificial neural network. The artificial neural network may be pre-trained to output the aneurysm point of the aneurysm region from the vessel mesh.

In some embodiments, the second electronic device 200 may verify the input point(s). For example, if the three input points are located on a single straight line, the second electronic device 200 may request re-entry of at least one of the input points. Similarly, if the two input points and the feature point are located on a single straight line, the second electronic device 200 may request re-entry of at least one of the input points.

The second electronic device 200 may generate an aneurysm region based on the separating plane (S130). The second electronic device 200 may determine one of the two regions separated by the separating plane as the aneurysm region. In some embodiments, the second electronic device 200 may determine the region in which the aneurysm point is located among the two regions separated by the separating plane as the aneurysm region.

In some embodiments, the second electronic device 200 may determine the aneurysm region based on the normal vector of the separating plane. For example, the second electronic device 200 may calculate the dot product of the normal vector of the separating plane and a verification vector. The verification vector may be defined by one input point and the aneurysm point. If the second user has input a plurality of input points, the second electronic device 200 may select one of the plurality of input points. If the result of the dot product is greater than or equal to 0, the second electronic device 200 may determine the region toward which the normal vector of the separating plane is directed as the aneurysm region. If the result of the dot product is less than 0, the second electronic device 200 may reverse the direction of the separating plane and determine the region toward which the reversed normal vector is directed as the aneurysm region. That is, the reversion of the separating plane make its normal vector reversed.

The second user may modify the separating plane. For example, the second user may modify the separating plane by adjusting the position of at least one input point on the second electronic device 200. Compared to conventional methods where a plane is generated at an arbitrary position in a three-dimensional coordinate system and then translated or rotated, the second electronic device 200 according to an embodiment may provide an interface that allows the second user to generate the separating plane more easily and precisely.

FIG. 3 illustrates a method of generating a separating plane on a vessel mesh by an electronic device according to an embodiment.

Referring to FIG. 3, an electronic device (e.g., the second electronic device 200 in FIG. 1) according to an embodiment may generate a vessel mesh (VMES). For example, the electronic device may obtain a plurality of input images from an imaging device and generate the vessel mesh (VMES) by applying an algorithm to the plurality of input images.

The electronic device may receive three input points (INP1, INP2, INP3). A user (for example, a medical professional) may input the input points (INP1, INP2, INP3) while viewing the vessel mesh (VMES) displayed by the electronic device.

The electronic device may generate a vessel mesh (VMES) from the plurality of input images transmitted by the imaging device, which captures the patient from various angles, thereby allowing the vessel mesh (VMES) to be displayed from various angles. That is, the electronic device may provide an interface that enables the user to observe the vessel mesh (VMES) from various angles. The electronic device may provide an interface that allows the vessel mesh (VMES) to be moved and/or rotated. The user may observe the vessel mesh (VMES) while changing the viewpoint through a display of the electronic device.

The electronic device may display the input points (INP1, INP2, INP3) based on a camera viewpoint displaying the vessel mesh (VMES) and the user input. For example, the electronic device may display the vessel mesh (VMES) from a first viewpoint through the display, and the user may input a first point into the electronic device on the vessel mesh (VMES) displayed from the first viewpoint. The electronic device may determine the point where the straight line connecting the first viewpoint and the first point first contacts the vessel mesh (VMES) as a first input point (INP1). The electronic device may display the first input point (INP1). Similarly, the electronic device may determine and display a second input point (INP2) and a third input point (INP3). In some embodiments, the user may input a second point and a third point from viewpoints different from the first viewpoint (for example, a second viewpoint and a third viewpoint, respectively), and the electronic device may determine the second input point (INP2) and the third input point (INP3) based on the corresponding viewpoints.

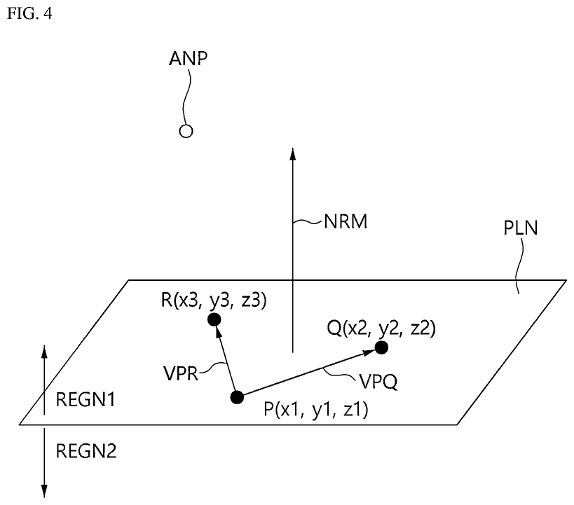

The electronic device may generate a separating plane in the three-dimensional space based on the input points (INP1, INP2, INP3). The electronic device may generate the separating plane passing through the input points (INP1, INP2, INP3). The separating plane may be used to distinguish between a parent artery and an aneurysm region. That is, the electronic device may generate the separating plane and determine one of the two regions separated by the separating plane as the aneurysm region. The configuration in which the electronic device determines the aneurysm region is described later with reference to FIGS. 4 to 8.

The electronic device may modify at least one of the input points (INP1, INP2, INP3) and regenerate the separating plane based on the modified input point. For example, the electronic device may modify the position of at least one of the input points (INP1, INP2, INP3) according to user input. Modifying the position of the input points (INP1, INP2, INP3) by the electronic device may be understood as modifying the coordinates.

The user may instruct the electronic device to modify the first input point (INP1) to a fourth point. The electronic device may determine the fourth input point based on the fourth point and replace the first input point (INP1) with the fourth input point. The electronic device may regenerate the separating plane based on the second input point (INP2), the third input point (INP3), and the fourth input point. For convenience of explanation, the electronic device has been described as modifying the first input point (INP1), but the same explanation applies to the second input point (INP2) and the third input point (INP3).

In this way, the electronic device may provide an interface that allows the user to generate the separating plane easily and precisely.

FIG. 4 illustrates a method of generating a plane by an electronic device according to an embodiment.

Referring to FIG. 4, an electronic device according to an embodiment may obtain input points (P, Q, R). The x, y, z coordinates of the input points (P, Q, R) may be (x1, y1, z1), (x2, y2, z2), and (x3, y3, z3), respectively. The electronic device may generate a plane (PLN) based on the input points (P, Q, R). The plane (PLN) may be a separating plane for separating (or isolating) the aneurysm region. The electronic device may generate a normal vector (NRM) of the plane (PLN) based on the input points (P, Q, R). For example, the electronic device may generate a first vector (VPQ) based on a first input point (P) and a second input point (Q). The electronic device may generate a second vector (VPR) based on the first input point (P) and the third input point (R). The electronic device may generate the normal vector (NRM) by performing a cross product of the first vector (VPQ) and the second vector (VPR).

The plane (PLN) generated by the electronic device may satisfy Equation 1.

$$a * x + b * y + c * z + d = 0 \qquad [ Equation 1 ]$$

Herein, a, b, and c may indicate x, y, and z coordinates of the normal vector (NRM), respectively. That is, coordinates of the normal vector (NRM) may be expressed as (a, b, c). Further, d may indicate a constant and may be determined by the input points (P, Q, R). In some embodiments, d may be defined as a value obtained by multiplying −1 by dot product of a third vector, which is defined from the origin to the first input point (P), and the normal vector (NRM).

The electronic device may determine the direction in which the normal vector (NRM) is pointing as the aneurysm region. For example, the vessel mesh may be divided into a first region (REGN1) and a second region (REGN2) by the plane (PLN). The electronic device may determine the first region (REGN1), which is enclosed by the vessel mesh and the plane (PLN), as the aneurysm region.

The electronic device may verify the determined aneurysm region. In some embodiments, the electronic device may determine whether an aneurysm point (ANP) is present in the first region (REGN1) corresponding to the normal vector (NRM). The aneurysm point (ANP) is a point located on the aneurysm region, which may be determined based on user input or inference from an artificial neural network. The electronic device may determine that the verification is successful if the aneurysm point (ANP) exists in the first region (REGN1). The electronic device may determine that the verification has failed if the aneurysm point (ANP) does not exist in the first region (REGN1).

In some embodiments, the electronic device may define a fourth vector based on the input points (P, Q, R) and the aneurysm point (ANP) and calculate the dot product of the fourth vector and the normal vector (NRM). In an example, the electronic device may define the fourth vector from one of the input points (P, Q, R) to the aneurysm point (ANP). In another example, the electronic device may define the fourth vector from the centroid (or center of mass) of the input points (P, Q, R) to the aneurysm point (ANP). In another example, the electronic device may define the fourth vector from the midpoint between two of the input points (P, Q, R) to the aneurysm point (ANP). However, the embodiment is not limited thereto, and the electronic device may define the fourth vector from any arbitrary point on the plane (PLN) to the aneurysm point (ANP). The electronic device may determine that the verification is successful if the dot product is greater than or equal to 0. The electronic device may determine that the verification has failed if the dot product is less than 0.

If the verification fails, the electronic device may reverse (or flip) the plane (PLN). The reversed plane may satisfy Equation 2.

$$a * x + b * y + c * z - d = 0 \\ - a * x - b * y - c * z + d = 0 \qquad [ Equation 2 ]$$

Herein, −a, −b, and −c may indicate the x, y, and z coordinates of the normal vector of the reversed (or flipped) plane, respectively. That is, coordinates of the normal vector of the reversed plane may be expressed as (−a, −b, −c), and its direction may be opposite to that of the normal vector (NRM). Further, d may indicate a constant and may be determined by the input points (P, Q, R). In some embodiments, d may be defined as a value obtained by multiplying −1 by dot product of the third vector, which is defined from the origin to the first input point (P), and the normal vector of the reversed plane.

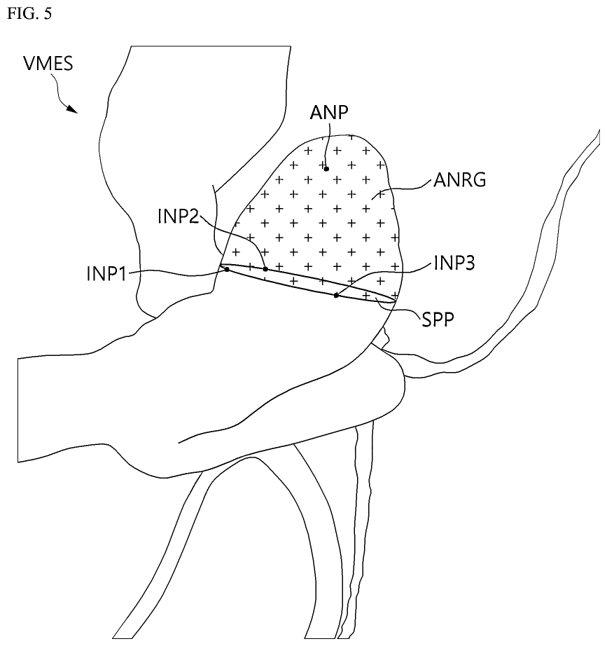

FIG. 5 illustrates a method of generating an aneurysm region on a vessel mesh by an electronic device according to an embodiment.

Referring to FIG. 5, the electronic device according to an embodiment may generate a vessel mesh (VMES) and generate at least one of the aneurysm point (ANP) and the input points (INP1, INP2, INP3) based on user input or inference from an artificial neural network. The electronic device may generate a separating plane (SPP) passing through the input points (INP1, INP2, INP3). The separating plane (SPP) may divide the vessel mesh (VMES) into a first region and a second region. At this time, one side of the separating plane (SPP) may be referred to as the first region, and the other side as the second region.

The electronic device may determine an aneurysm region (ANRG) based on the vessel mesh (VMES) and the separating plane (SPP). The aneurysm region (ANRG) may be defined as a three-dimensional shape with volume or may refer to the surface of a two-dimensional mesh. In an example, the electronic device may determine the first region enclosed by the vessel mesh (VMES) and the separating plane (SPP) as the aneurysm region (ANRG). The first region may be a three-dimensional shape with volume. In another example, the electronic device may determine the surface area of the vessel mesh (VMES) in the first region, excluding the separating plane (SPP), as the aneurysm region (ANRG). By highlighting and displaying the aneurysm region (ANRG), the electronic device may provide an environment where the user can better analyze the aneurysm region (ANRG) corresponding to the area of interest on the vessel mesh (VMES).

The electronic device may verify the aneurysm region (ANRG) based on the normal vector of the separating plane (SPP) and the aneurysm point (ANP). For example, the electronic device may determine that the verification is successful if the normal vector of the separating plane (SPP) is directed toward the aneurysm point (ANP). The electronic device may determine that the verification has failed if the normal vector of the separating plane (SPP) is not directed toward the aneurysm point (ANP). In some embodiments, the electronic device may define a first vector based on the input points (INP1, INP2, INP3) and the aneurysm point (ANP) and perform the verification based on the dot product of the first vector and the normal vector.

If the verification is successful, the electronic device may maintain the aneurysm region (ANRG); and if the verification fails, the electronic device may reverse (or flip) the separating plane (SPP). The electronic device may reverse the separating plane (SPP) upon verification failure and determine the second region, instead of the first region, as the aneurysm region (ANRG).

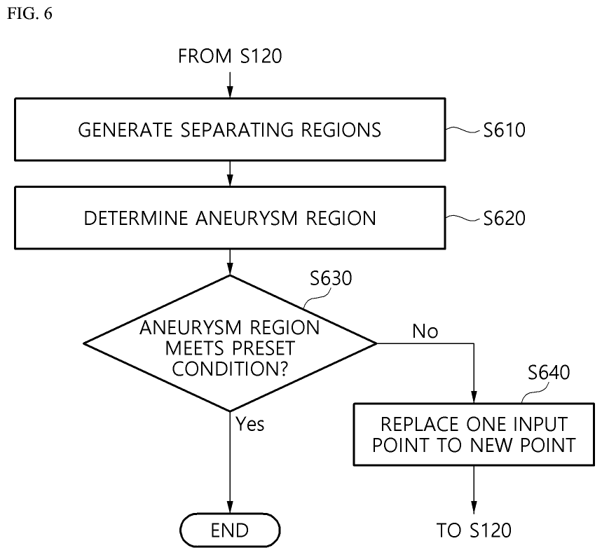

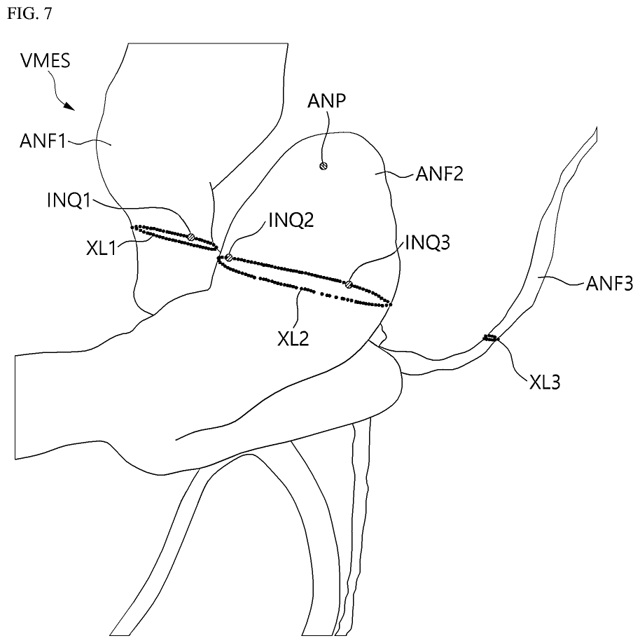

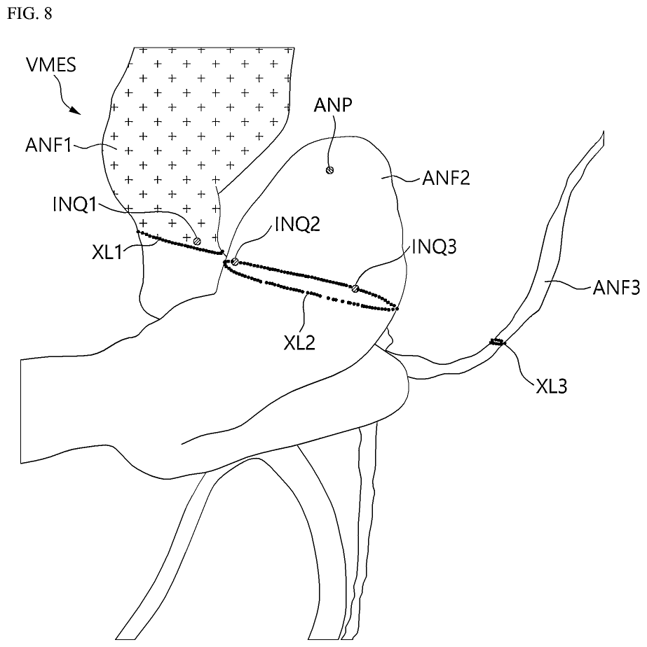

FIG. 6 is a flowchart of a method for generating an aneurysm region by an electronic device according to an embodiment, and FIGS. 7 and 8 illustrate a method for generating an aneurysm region on a vessel mesh by an electronic device according to an embodiment.

Referring to FIGS. 6 and 7, the electronic device according to an embodiment may receive the input points (INQ1, INQ2, INQ3) and generate a separating plane based on the input points (INQ1, INQ2, INQ3) (S120). In some embodiments, the generated separating plane may exist as an entity extending in a three-dimensional space and may divide the vessel mesh (VMES) multiple times. For example, the separating plane generated based on the input points (INQ1, INQ2, INQ3) may generate intersection lines (XL1, XL2, XL3) where the separating plane contacts the vessel mesh (VMES). The input points (INQ1, INQ2, INQ3) may be located on the intersection lines (XL1, XL2, XL3). For example, a first input point (INQ1) may be located on a first intersection line (XL1), and second and third input points (INQ2, INQ3) may be located on a second intersection line (XL2). Although three intersection lines (XL1, XL2, XL3) are shown in FIG. 7 for convenience of explanation, one or more additional intersection lines that contact the separating plane may exist depending on the embodiments.

In some embodiments, the electronic device may determine whether the input points (INQ1, INQ2, INQ3) are located on the same intersection line. The electronic device may request the user to re-enter at least one of the input points (INQ1, INQ2, INQ3) if the input points (INQ1, INQ2, INQ3) are not located on the same intersection line. That is, in FIG. 7, the electronic device may request re-entry to the user as the input points (INQ1, INQ2, INQ3) are not located on the same intersection line. For example, the electronic device may determine that one of the points (e.g., INQ1) is located on a different intersection line (e.g., XL1) among the three input points (INQ1, INQ2, INQ3). The electronic device may suggest modifying at least one point. Entering, by user, three-dimensional points on a two-dimensional display without depth information may lead to unexpected errors. That is, the user may intend to input a point in the region of the aneurysm point (ANP), but an input point may be generated in another area, such as the first input point (INQ1), which is not in the aneurysm region. The electronic device according to an embodiment may suggest modifying the point if such an error occurs, thereby providing an environment that allows the user to more precisely determine the aneurysm region.

The electronic device may generate separating regions (ANF1, ANF2, ANF3) based on the separating plane (S610). The electronic device may determine the upper region among the areas separated by the separating plane as the separating regions (ANF1, ANF2, ANF3) based on the normal vector of the separating plane and the aneurysm point (ANP). The electronic device may generate a first separating region (ANF1) based on the first intersection line (XL1), a second separating region (ANF2) based on the second intersection line (XL2), and the third separating region (ANF3) based on a third intersection line (XL3) of the separating plane.

The electronic device may determine one of the separating regions (ANF1, ANF2, ANF3) as the aneurysm region (S620). In some embodiments, the electronic device may select one of the separating regions (ANF1, ANF2, ANF3) based on the input points (INQ1, INQ2, INQ3). The electronic device may calculate a first centroid of the input points (INQ1, INQ2, INQ3) and determine the region closest to the first centroid among the separating regions (ANF1, ANF2, ANF3) as the aneurysm region. In an example, the electronic device may determine the intersection line closest to the first centroid among the intersection lines (XL1, XL2, XL3) and determine the region corresponding to the selected intersection line among the separating regions (ANF1, ANF2, ANF3) as the aneurysm region. In another example, the electronic device may determine second centroids of the intersection lines (XL1, XL2, XL3), determine a third centroid closest to the first centroid among the second centroids, and determine the region corresponding to the third centroid as the aneurysm region. However, the embodiment is not limited thereto, and the electronic device may use various methods to determine the region closest to the first centroid.

In some embodiments, the electronic device may select one of the separating regions (ANF1, ANF2, ANF3) based on the input points (INQ1, INQ2, INQ3) and the aneurysm point (ANP). The electronic device may calculate a fourth centroid of the input points (INQ1, INQ2, INQ3) and the aneurysm point (ANP) and determine the region closest to the fourth centroid among the separating regions (ANF1, ANF2, ANF3) as the aneurysm region.

In some embodiments, the electronic device may select one of the separating regions (ANF1, ANF2, ANF3) based on the aneurysm point (ANP). The electronic device may determine a region among the separating regions (ANF1, ANF2, ANF3) in which the aneurysm point (ANP) is located as the aneurysm region.

The electronic device may determine whether the aneurysm region meets a preset condition (S630). In some embodiments, the electronic device may determine whether the aneurysm region includes the aneurysm point (ANP). The electronic device may determine that the preset condition is met if the aneurysm region includes the aneurysm point (ANP). The electronic device may determine that the preset condition is not met, if the aneurysm region does not include the aneurysm point (ANP).

Referring to FIG. 8, the electronic device according to an embodiment may determine the first separating region (ANF1) as the aneurysm region. The aneurysm point (ANP) may be located on the second separating region (ANF2) in the vessel mesh (VMES). Thus, the electronic device may determine that the preset condition is not met because the first separating region (ANF1) does not include the aneurysm point (ANP).

If the aneurysm region does not meet the preset condition (S630, NO), the electronic device may replace one input point with a new input point (S640). The user may instruct the electronic device to modify one (e.g., INQ1) of the input points (INQ1, INQ2, INQ3). The electronic device may generate a separating plane based on the new input point (S120).

If the aneurysm region meets the preset condition (S125, YES), the electronic device may maintain the aneurysm region based on the separating plane.

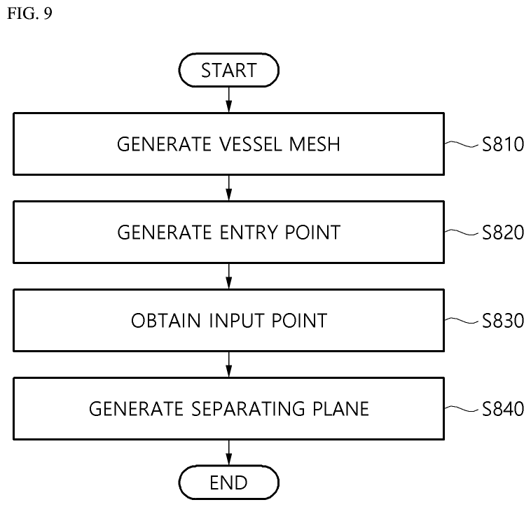

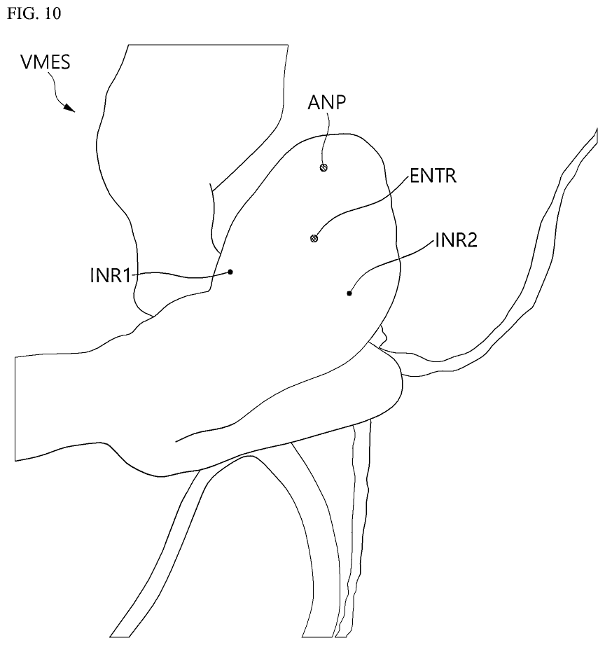

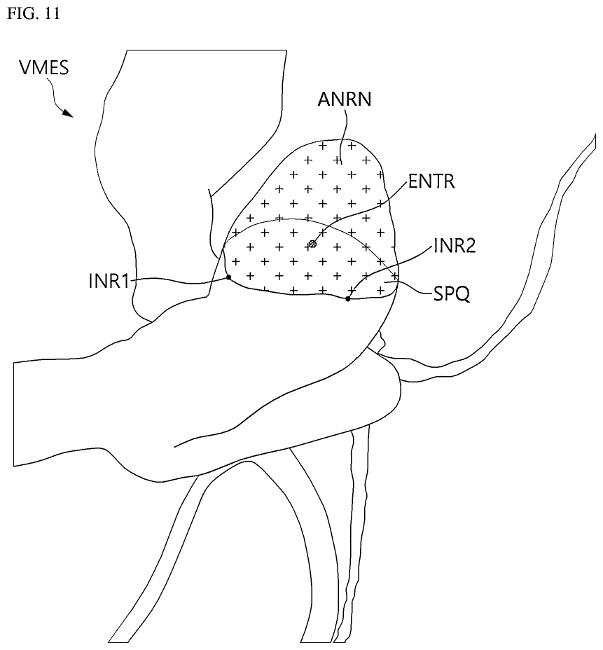

FIG. 9 is a flowchart illustrating a method for generating a separating plane on a vessel mesh by an electronic device according to an embodiment, and FIGS. 10 and 11 are illustrate a method for generating a separating plane on a vessel mesh by an electronic device according to an embodiment.

Referring to FIGS. 9 and 10, an electronic device according to an embodiment may generate a vessel mesh (VMES) (S810). The electronic device may receive a plurality of images captured by an imaging device. The electronic device may generate the vessel mesh (VMES) by applying marching cube algorithm to the plurality of images. The vessel mesh (VMES) may be data representing blood vessels in three dimensions. The electronic device may display the vessel mesh (VMES) through a display. The user can observe the vessel mesh (VMES) while changing viewpoint. For example, the user can change the viewpoint by moving and/or rotating the vessel mesh (VMES) while viewing the vessel mesh (VMES) through the display.

The electronic device may generate an entry point (ENTR) (S820). The entry point (ENTR) may refer to a point where a blood vessel leads into an aneurysm. The electronic device may generate a centerline within the vessel mesh (VMES) and generate the entry point (ENTR) based on the centerline. The centerline may include a vessel centerline that connects blood vessels and an aneurysm centerline that connects an aneurysm to a blood vessel. The electronic device may determine the entry point (ENTR) based on the aneurysm centerline. For example, the electronic device may determine one of the points on the aneurysm centerline as the entry point (ENTR).

The electronic device may generate a voronoi diagram based on the vessel mesh (VMES). The electronic device may generate a plurality of inscribed spheres within the vessel mesh (VMES) based on the voronoi diagram. The voronoi diagram may define points associated with the vessel mesh (VMES), the radius of the inscribed spheres, and the like. The electronic device may determine points where the sum of the radii of the plurality of inscribed spheres is maximized and may determine a line connecting the points as the centerline. The points may represent the centers of the inscribed spheres. In some embodiments, the electronic device may generate the centerline using VMTK (Vascular Modeling Toolkit) library.

The electronic device may generate a vessel centerline. The electronic device may detect an open vessel within the vessel mesh (VMES). The open vessel may refer to a blood vessel that is not occluded. The electronic device may generate a vessel centerline that reaches from one open vessel to another open vessel in the vessel mesh. The electronic device may generate a plurality of inscribed spheres along the path from one open vessel to another open vessel. The electronic device may generate the inscribed spheres so that the sum of the radii of the inscribed spheres is maximized, and may generate the vessel centerline by connecting center points of the inscribed spheres. Specifically, when a first open vessel is the start point and a second open vessel is the end point, the electronic device may determine points where the sum of the radii of the inscribed spheres between the first open vessel and the second open vessel is maximized as the vessel centerline. The electronic device may perform interpolation when generating the vessel centerline by connecting the points.

The electronic device may generate a tube mesh based on the vessel centerline. The tube mesh may be a tube-shaped mesh corresponding to a parent artery. The electronic device may generate the tube mesh based on the inscribed spheres of the vessel centerline. For example, the electronic device may generate the tube mesh based on center points of the vessel centerline and radius information corresponding to each center point.

The electronic device may determine an aneurysm center point based on the aneurysm point (ANP) and the tube mesh. The aneurysm center point can be understood as a point that is located within the aneurysm region and represents the aneurysm region. In some embodiments, the electronic device may determine the aneurysm point (ANP) based on user input. In some embodiments, the electronic device may determine the aneurysm point (ANP) using an artificial neural network trained to output an aneurysm point (e.g., ANP) from the vessel mesh (VMES). In some embodiments, the artificial neural network may be trained to output an aneurysm center point from the vessel mesh (VMES).

The electronic device may generate a plurality of first rays within the vessel mesh (VMES), starting from the aneurysm point (ANP) as a start point. The electronic device may determine first points where the plurality of first rays intersect with the tube mesh.

The electronic device may determine the aneurysm center point based on the first points. For example, the electronic device may determine the center of the aneurysm point (ANP) and the first points as a first center point. The electronic device may generate a plurality of second rays within the vessel mesh (VMES), starting from the first center point as a start point. The electronic device may determine the second points where the plurality of second rays intersect with the tube mesh.

The electronic device may determine the center of the first center point and the second points as a second center point. If the distance between the first center point and the second center point is less than (or less than or equal to) a predetermined distance, the electronic device may determine the second center point as the aneurysm center point.

If the distance between the first center point and the second center point is greater than or equal to the predetermined distance, the electronic device may determine a third center point based on the second center point. If the distance between the second center point and the third center point is less than the predetermined distance, the electronic device may determine the third center point as the aneurysm center point. In this manner, the electronic device may continue to determine a center point until the distance between a previous center point and a current center point is less than the predetermined distance.

The electronic device may generate the aneurysm centerline based on an open vessel and the aneurysm center point. The electronic device may generate an aneurysm centerline extending from one open vessel to the aneurysm center point in the vessel mesh (VMES). The electronic device may generate a plurality of inscribed spheres along the path from one open vessel to the aneurysm center point. The electronic device may generate the inscribed spheres so that the sum of the radii of the inscribed spheres is maximized and may generate the aneurysm centerline by connecting center points of the inscribed spheres. Specifically, when the aneurysm is the start point and an open vessel is the end point, the electronic device may determine points where the sum of the radii of the inscribed spheres between the aneurysm and the open vessel is maximized as the aneurysm centerline. The electronic device may perform interpolation when generating the aneurysm centerline by connecting the points.

In some embodiments, the electronic device may also generate an aneurysm centerline based on the open vessel and the aneurysm point (ANP).

The electronic device may determine the entry point (ENTR) based on the aneurysm centerline and the tube mesh. The electronic device may determine third points where the aneurysm centerline intersects the tube mesh. That is, the aneurysm centerline may intersect the tube mesh at the third points. The electronic device may determine the center of the third points as the entry point (ENTR).

The configuration of the electronic device generating the entry point (ENTR) is referenced in Korean Patent Application No. 10-2024-0087635 filed in the Korean Intellectual Property Office on Jul. 3, 2024, and may be incorporated herein in its entirety. Additionally, the electronic device is not limited to the aforementioned example and may determine a point on the aneurysm centerline as the entry point (ENTR) using various methods.

The electronic device may obtain input points (INR1, INR2) (S830). The user may input a first point and a second point into the electronic device using an input/output device. The electronic device may generate straight lines from current viewpoint where the vessel mesh (VMES) is displayed toward the first point and the second point, respectively, and determine points where the straight lines first contact the vessel mesh (VMES) as the input points (INR1, INR2).

The user may input the input points (INR1, INR2) while maintaining or changing the viewpoint. In an example, the user may input a first input point (INR1) and a second input point (INR2) into the electronic device while viewing the vessel mesh (VMES) from a first viewpoint. In another example, the user may input the first input point (INR1) while viewing the vessel mesh (VMES) from the first viewpoint and then change the viewpoint to the second viewpoint to input the second input point (INR2). At this time, the user may change the viewpoint by moving and/or rotating the vessel mesh (VMES).

In some embodiments, the electronic device may verify the input points (INR1, INR2). For example, if the entry point (ENTR) and the input points (INR1, INR2) are located on a single straight line, the electronic device may request re-entry of at least one of the input points (INR1, INR2).

Although FIG. 10 describes a configuration where the electronic device obtains two input points (INR1, INR2) for convenience of explanation, the embodiment is not limited thereto, and the electronic device may obtain one or three input points.

Referring to FIGS. 9 to 11, the electronic device may generate a separating plane (SPQ) based on the entry point (ENTR) and the input points (INR1, INR2) (S840). For example, the electronic device may generate the separating plane (SPQ) passing through the entry point (ENTR) and the input points (INR1, INR2).

In some embodiments, when the user inputs one input point into the electronic device, the electronic device may generate the separating plane (SPQ) based on the one input point and an aneurysm vector. The electronic device may generate the separating plane (SPQ) that passes through the input point, with the aneurysm vector as the normal vector. The aneurysm vector may be a vector directed from the entry point (ENTR) to the aneurysm point (ANP).

The electronic device may generate an aneurysm region (ANRN) based on the separating plane (SPQ). The electronic device may determine one of the two regions separated by the separating plane (SPQ) as the aneurysm region (ANRN). The electronic device may determine the other one of the two regions as a parent artery region. The previously described method of determining the aneurysm region (ANRN) by the electronic device applies similarly here. Therefore, redundant explanations are omitted.

The user may modify the separating plane (SPQ) by adjusting the position of at least one of the input points (INR1, INR2) in the electronic device. Compared to conventional methods where a plane is generated at an arbitrary position in a three-dimensional coordinate system and then translated or rotated, an electronic device according to an embodiment may provide an interface that allows the user to generate the separating plane (SPQ) easily and precisely.

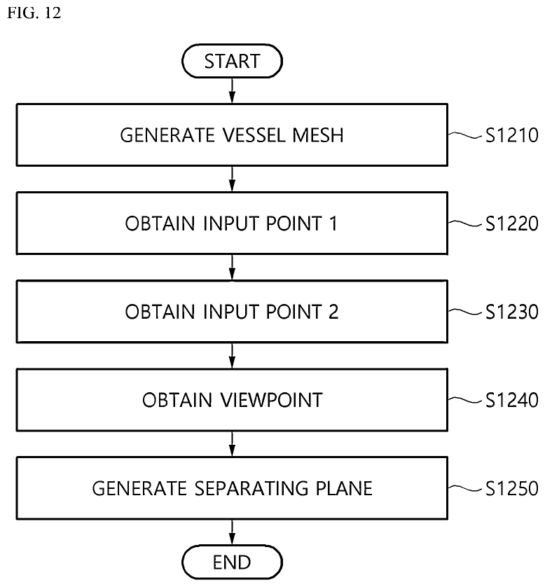

FIG. 12 is a flowchart illustrating a method for generating a separating plane by an electronic device according to an embodiment.

Referring to FIG. 12, an electronic device according to an embodiment may generate a vessel mesh (S1210). The electronic device may obtain a plurality of input images from an imaging device and may generate the vessel mesh from the plurality of input images. The configuration of generating the vessel mesh by the electronic device may be similarly applied to the description explained with reference to FIG. 2 or FIG. 9. Therefore, redundant descriptions are omitted.

The electronic device may obtain a first input point (S1220). The user may select a first point on the vessel mesh through the display of the electronic device. The electronic device may obtain the first input point based on a first viewpoint and the first point on the vessel mesh. For example, the electronic device may generate a first line based on the first viewpoint and the first point, and may determine a point where the first line first contacts the vessel mesh as the first input point.

The electronic device may obtain a second input point (S1230). In response to selection of a second point by the user, the electronic device may obtain the second input point based on a second viewpoint and the second point at the time when the second point is selected. Depending on the embodiments, the first viewpoint and the second viewpoint may be the same or different.

The electronic device may obtain a viewpoint (S1240). In some embodiments, the electronic device may obtain the first viewpoint corresponding to the first input point or the second viewpoint corresponding to the second input point.

In some embodiments, the electronic device may determine camera viewpoint that best shows an aneurysm region (e.g., ANRN in FIG. 11) among the received imaging data. The camera viewpoint may refer to the location of the imaging device that is the source of the imaging data. The electronic device may determine camera viewpoints based on the normal vector. In some embodiments, the electronic device may determine camera viewpoints based on the normal vector of the separating plane (e.g., SPQ in FIG. 11). In some embodiments, the electronic device may determine camera viewpoints such that the angle between the vector pointing towards an aneurysm center point (or aneurysm entry point) from the camera viewpoint and the normal vector falls within a predetermined angle range. For example, the predetermined angle range may be set to have a certain angle margin around 90 degrees. In other words, the electronic device may determine the camera viewpoints where the angle between the vector pointing towards the aneurysm center point (or aneurysm entry point) from the camera viewpoint and the separating plane is approximately 0 degrees.

The electronic device may determine the scores of the determined camera viewpoints. The electronic device may determine the scores based on the aneurysm region. In some embodiments, the electronic device may determine the scores based on the degree of overlap between the aneurysm region and other blood vessels. The electronic device may separate the vessel mesh into the aneurysm region and a blood vessel region using the separating plane. The electronic device may calculate the area of obscured part of the aneurysm region, which is obscured by the blood vessel region, and determine the scores based on the area. In some embodiments, the electronic device may calculate the percentage (or ratio) of the aneurysm region that is obscured by the blood vessel region and determine the scores based on the percentage.

In some embodiments, the electronic device may determine the scores based on the degree of overlap between the neck of the aneurysm region and other blood vessels. That is, the electronic device may calculate the area of obscured part of the neck of the aneurysm region and determine the scores based on the area. The neck of the aneurysm region may be obscured by the blood vessel region.

In some embodiments, the electronic device may determine the scores based on how well the neck of the aneurysm region is visible. For example, the electronic device may determine the scores based on the angle between the vector pointing towards the aneurysm center point (or aneurysm entry point) from the camera viewpoint and the normal vector of the separating plane. The electronic device may determine the scores based on trigonometric functions (e.g., sine function) and the angle.

In other words, the electronic device may assign relatively higher scores to camera viewpoints where the area of the aneurysm region obscured by the blood vessel region is smaller, the area of the neck of the aneurysm region obscured by the blood vessel region is smaller, and the angle is closer to 90 degrees.

The electronic device may generate the separating plane based on the first input point, the second input point, and the viewpoint (S1250). The electronic device may generate the separating plane passing through the first input point, the second input point, and the viewpoint.

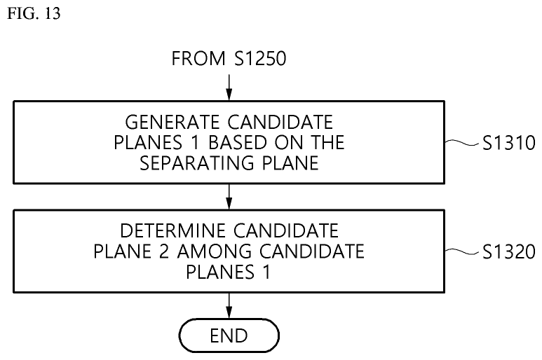

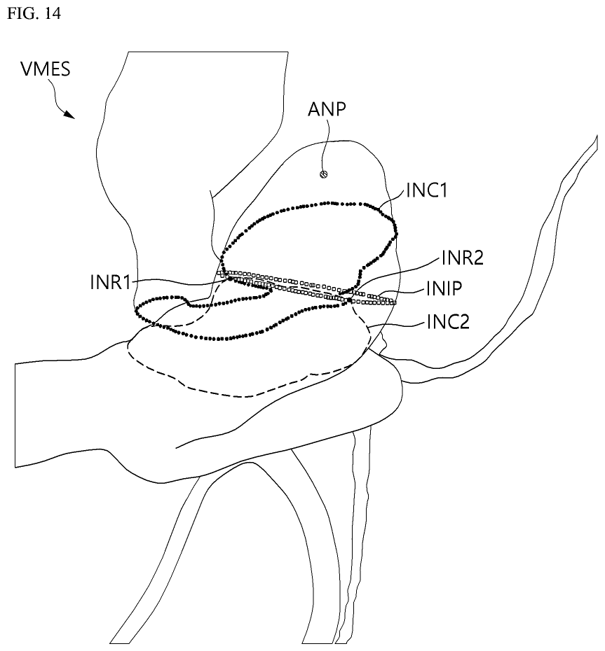

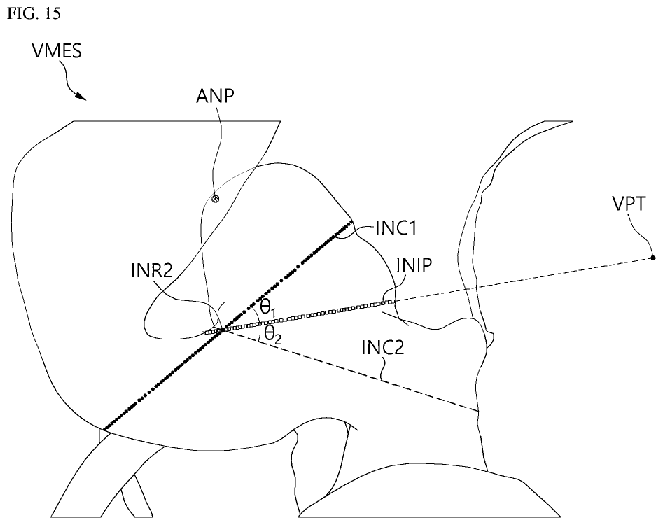

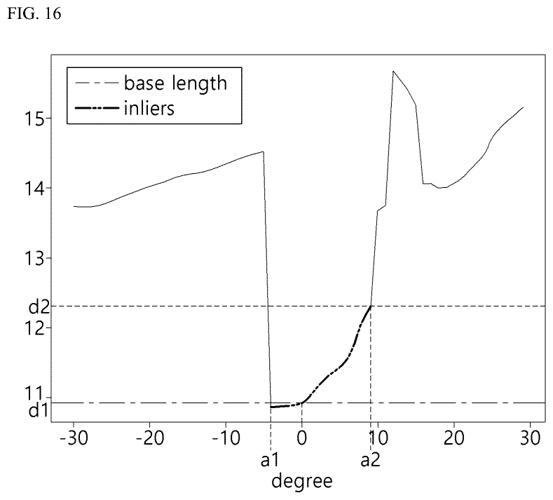

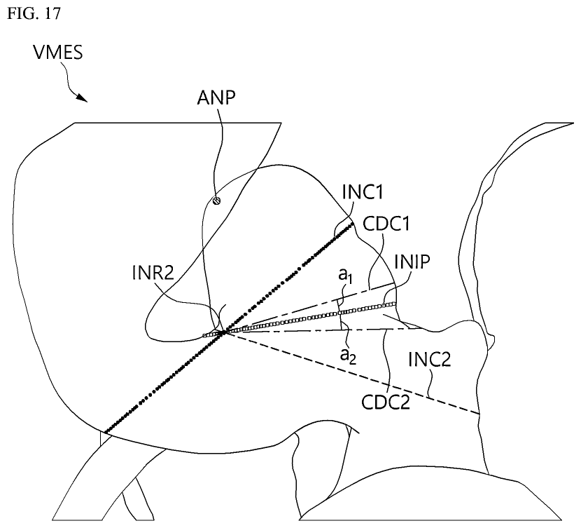

FIG. 13 is a flowchart illustrating a method for generating a separating plane by an electronic device according to an embodiment, FIGS. 14 and 15 illustrate a method for generating a separating plane by an electronic device according to an embodiment, FIG. 16 is a graph showing the length of the intersection line according to a rotation angle of a separating plane according to an embodiment, and FIG. 17 illustrate a method for generating a separating plane by an electronic device according to an embodiment.

Referring to FIGS. 13 to 15, the electronic device according to an embodiment may generate a separating plane based on a first input point (INR1), a second input point (INR2), and a viewpoint (VPT). The electronic device may generate an intersection line (INIP) where the separating plane and the vessel mesh (VMES) meet.

The electronic device may generate first candidate planes based on the separating plane (S1310). In some embodiments, the electronic device may generate the first candidate planes by rotating the separating plane. For example, the electronic device may rotate the separating plane using the line connecting the first input point (INR1) and the second input point (INR2) as a rotation axis. The electronic device may generate a first candidate plane positioned above a first reference angle (θ₁) relative to the separating plane and a first candidate plane positioned below a second reference angle (θ₂) relative to the separating plane. The electronic device may generate an intersection line (INC1) where a first candidate plane at the first reference angle (θ₁) and the vessel mesh (VMES) meet, and may generate an intersection line (INC2) where a first candidate plane at the second reference angle (θ₂) and the vessel mesh (VMES) meet. Herein, the configuration that a first candidate plane is positioned above/below the first and second reference angles (θ₁, θ₂) relative to the separating plane may represent that the separating plane is rotated by the reference angles (θ₁, θ₂) as the rotation axis to generate the first candidate planes.

In some embodiments, the electronic device may generate the first candidate planes by parallel translation of the separating plane. For example, the electronic device may move the separating plane in the direction of the normal vector or in the reverse direction of the normal vector. The electronic device may generate planes within a predetermined distance range from the separating plane as the first candidate planes. In this manner, the electronic device may generate the first candidate planes from the separating plane using various methods.

The electronic device may determine a second candidate plane from among the first candidate planes (S1320). In some embodiments, the electronic device may determine the second candidate plane based on the length of the intersection line (INIP) of the separating plane.

FIG. 16 is a graph showing the length of the intersection line (or length of cross section) generated by rotating the separating plane by a predetermined angle. In the graph, the separating plane is a reference and is at 0 degrees. Referring to FIG. 16 together, the electronic device may determine the length d1 of the intersection line (INIP) of the separating plane as a base length (or a reference length). The electronic device may determine a boundary length (d2) based on the base length (d1). For example, the electronic device may determine the boundary length (d2) by adding or multiplying a reference value to the base length (d1). Depending on the embodiments, the reference value may be determined differently. The electronic device may determine a separating plane with an intersection line length equal to or less than (or less than) the boundary length (d2) as the second candidate plane. The second candidate plane may be within the range of a first angle (a₁) to a second angle (a₂) relative to the separating plane. The first angle (a₁) may be a negative real number, and the second angle (a₂) may be a positive real number.

Referring to FIG. 17 together, the electronic device may generate an intersection line (CDC1) corresponding to the first angle (a₁) and an intersection line (CDC2) corresponding to the second angle (a₂). The electronic device may display the second candidate plane that exists within the range of the first angle (a₁) to the second angle (a₂). There may be a plurality of second candidate planes.

In some embodiments, the electronic device may calculate the score of the second candidate plane. For example, the electronic device may calculate the score based on at least one of a first volume of an aneurysm region, a second volume of a parent artery region, and the perimeter of the intersection line between the separating plane and the vessel mesh. The first volume of the aneurysm region may refer to the volume of the area obtained by the vessel mesh and the separating plane. The second volume of the parent artery region may refer to the volume of the area obtained by a parent artery mesh, clipping points, and the separating plane. The clipping point is a point on the vessel centerline and may represent the boundaries of the aneurysm region. For example, the aneurysm region may begin at a first clipping point and may end at a second clipping point. For example, the electronic device may determine clipping points at points along the vessel centerline based on the tube mesh and the aneurysm center point (or aneurysm entry point or aneurysm point).

In some embodiments, the electronic device may calculate the score of the second candidate plane based on Equation 3.

$${SCR}_{CP 1} = V 1 * w + V 2 * ( 1 - w ) \qquad [ Equation 3 ]$$

Herein, SCR_{CP1} may indicate the score of a second candidate plane, V1 may indicate a first volume, w may indicate a weight, and V2 may indicate a second volume.

In some embodiments, the electronic device may calculate the score of the second candidate plane based on Equation 4.

$${SCR}_{CP 1} = V 1 * {w}_{1} + V 2 * {w}_{2} \qquad [ Equation 4 ]$$

Herein, SCR_{CP1} may indicate the score of a second candidate plane, V1 may indicate a first volume, w₁ may indicate a first weight, V2 may indicate a second volume, and w₂ may indicate a second weight.

In some embodiments, the electronic device may calculate the score of the second candidate plane based on Equation 5 or Equation 6.

$${SCR}_{CP 1} = V 1 * w + V 2 * ( 1 - w ) - CCF \qquad [ Equation 5 ]$$

$${SCR}_{CP 1} = \frac{V 1 * w + V 2 * ( 1 - w )}{CCF} \qquad [ Equation 6 ]$$

Herein, SCR_{CP1} may indicate the score of a second candidate plane, V1 may indicate a first volume, w may indicate a weight, V2 may indicate a second volume, and CCF may indicate the perimeter of the intersection line between the second candidate plane and the vessel mesh.

However, the embodiment is not necessarily limited to the examples mentioned above. The electronic device may calculate the score using at least one of the first volume, the second volume, and the perimeter, along with various weights.

The electronic device may determine whether the score of the second candidate plane exceeds a threshold. The threshold may be a value preset by the user. In some embodiments, the signs of the right-hand terms in the aforementioned Equations 3 to 6 may be reversed. In this case, the electronic device may determine whether the calculated score is below the threshold.

The electronic device may determine the second candidate plane as the separating plane to obtain the aneurysm region if the score of the second candidate plane exceeds the threshold. In some embodiments, the user may select the second candidate plane, and the electronic device may change the separating plane based on the user's selection. The electronic device may determine an area where the aneurysm point (ANP) is located among the separated regions as the aneurysm region.

In some embodiments, the electronic device may calculate the scores of all second candidate planes generated based on the separating plane and the boundary length (d2), and determine a second candidate plane with the highest score as a final separating plane.

The electronic device, once the aneurysm region is obtained, may perform a quantitative analysis of the aneurysm region. For example, the electronic device may determine parameters such as maximal neck diameter, minimal neck diameter, height, width, and angle for the aneurysm region. Herein, the maximal neck diameter may indicate the longest line in the neck of the separating plane, and the minimal neck diameter may indicate the longest line perpendicular to the maximal neck diameter. The neck of the separating plane may indicate a closed loop defined by the intersection line of the separating plane and the vessel mesh (VMES). The height may include height 1, height 2, and height (ortho). Height 1 may indicate the longest line extending from the center of mass of the separating plane toward the aneurysm region, height 2 may indicate the longest line extending from the midpoint of the maximal neck diameter toward the aneurysm region, and height (ortho) may indicate the line extending to the point in the aneurysm region that is farthest from the separating plane. The width may indicate the longest line perpendicular to each height, and the angle may indicate the angle formed between the separating plane and each height.

As described above, the electronic device may provide an interface to the user that allows for the rapid and precise generation of the separating plane. Through the electronic device, the user can reduce errors in generating the separating plane and conveniently correct any errors that do occur, thereby enhancing the user experience.

In some embodiments, each component or a combination of two or more components described with reference to FIG. 1 to FIG. 17 may be implemented with digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, LED (light-emitting diode) monitor, OLED (organic LED) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. Accordingly, other implementations are within the scope of the following claims.

What is claimed is:

1. A method for generating an aneurysm region, comprising:
generating a vessel mesh;
obtaining at least one input point;
generating a separating plane based on the at least one input point; and
generating the aneurysm region based on the separating plane.

2. The method of claim 1, wherein the obtaining the at least one input point comprises:
receiving a first input point and a second input point from a user, and
wherein the generating the separating plane comprises:
obtaining a camera viewpoint corresponding to the first input point or the second input point; and
generating the separating plane based on the camera viewpoint, the first input point, and the second input point.

3. The method of claim 1, wherein the method further comprises:
obtaining an aneurysm point; and
determining an aneurysm entry point based on the aneurysm point, and
wherein the generating the separating plane comprises:
generating the separating plane based on the aneurysm entry point and the at least one input point.

4. The method of claim 3, wherein the determining the aneurysm entry point comprises:
generating a vessel centerline based on an open vessel of the vessel mesh;
generating a tube mesh based on the vessel centerline;
determining an aneurysm center point based on the aneurysm point and the tube mesh;
generating an aneurysm centerline based on the open vessel and the aneurysm center point; and
determining the aneurysm entry point based on the aneurysm centerline and the tube mesh.

5. The method of claim 4, wherein the obtaining the at least one input point comprises:
receiving a first input point and a second input point from a user, and
wherein the generating the separating plane comprises:
generating the separating plane based on the aneurysm entry point, the first input point, and the second input point.

6. The method of claim 4, wherein the obtaining the at least one input point comprises:
receiving a first input point from a user, and
wherein the generating the separating plane comprises:
generating a first vector directed from the aneurysm entry point toward the aneurysm center point; and
generating, as the separating plane, a plane passing through the first input point with the first vector as a normal vector.

7. The method of claim 1, wherein the method further comprises:
obtaining an aneurysm point, and
wherein the generating the aneurysm region comprises:
obtaining a first region and a second region separated by the separating plane; and
determining a region corresponding to the aneurysm point among the first region and the second region as the aneurysm region.

8. The method of claim 7, wherein the determining the region corresponding to the aneurysm point among the first region and the second region as the aneurysm region comprises:
obtaining a normal vector of the separating plane;
generating a first vector directed from a point on the separating plane toward the aneurysm point; and
determining the aneurysm region based on dot product of the normal vector and the first vector.

9. The method of claim 8, wherein the determining the aneurysm region based on dot product of the normal vector and the first vector comprises:
determining a direction in which a normal vector of the separating plane is directed as the aneurysm region if the dot product is equal to or greater than 0; and
inverting the separating plane and determining the direction in which a normal vector of the inverted separating plane is directed as the aneurysm region if the dot product is less than 0.

10. The method of claim 1, wherein the obtaining the at least one input point comprises:
obtaining a first input point, a second input point, and a third input point from a user, and
wherein the method further comprises:
generating an intersection line where the separating plane contacts the vessel mesh;
maintaining the separating plane if the first input point, the second input point, and the third input point are located on the same intersection line; and
requesting re-entry of the at least one input point if the first input point, the second input point, and the third input point are not located on the same intersection line.

11. The method of claim 1, wherein the generating the aneurysm region comprises:
generating first candidate planes based on the separating plane; and
generating the aneurysm region using a second candidate plane among the first candidate planes.

12. The method of claim 11, wherein the generating the first candidate planes comprises:
generating the first candidate planes by parallel translation of the separating plane in a direction of a normal vector of the separating plane by a predetermined distance range.

13. The method of claim 11, wherein the obtaining the at least one input point comprises:
receiving a first input point and a second input point from a user, and
wherein the generating the first candidate planes comprises:
determining a straight line connecting the first input point and the second input point as a rotation axis; and
generating the first candidate planes by rotating the separating plane by a predetermined angle range using the rotation axis.

14. The method of claim 13, wherein the generating the aneurysm region using the second candidate plane among the first candidate planes comprises:
calculating a base length of an intersection line of the separating plane;
determining a boundary length based on the base length; and
determining, as the second candidate plane, a plane among the first candidate planes whose intersection line with the vessel mesh has a length smaller than the boundary length.

15. The method of claim 14, wherein the generating the aneurysm region using the second candidate plane among the first candidate planes further comprises:
obtaining a plurality of second candidate planes;
calculating a score for each of the plurality of second candidate planes; and
selecting one of the plurality of second candidate planes based on the score.

16. The method of claim 15, wherein the calculating the score comprises:
calculating the score based on at least one of a first volume of the aneurysm region obtained by one of the second candidate planes, a second volume of a parent artery region obtained by the one of the second candidate planes, and a perimeter of the intersection line between the one of the second candidate planes and the vessel mesh.

17. An electronic device comprising:
a processor, and
a memory connected to the processor,
wherein the memory is configured to store a program,
wherein the processor is configured to execute the program, and
wherein when the program is executed by the processor, the steps of the method of claim 1 are implemented.
